(19) United States
(12) Patent Application Publication
(10) Pub. No.: US 2009/0186098 A1
(43) Pub. Date: Jul. 23, 2009

(54) SPORTS DRINK COMPOSITION

(76) Inventor: Jose Briceno, Liberty Township, OH (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 12/357,202

(22) Filed: Jan. 21, 2009

Related U.S. Application Data
(60) Provisional application No. 61/021,980, filed on Jan. 18, 2008.

(51) Int. Cl. A61K 33/20; A61K 38/02; A61K 33/00

(52) U.S. Cl. 424/661; 514/2; 424/722

(57) ABSTRACT

A composition having a high molecular weight carbohydrate with an average molecular weight greater than about 10,000. A hydrolyzed protein is also included in the composition, the protein having an average molecular weight less than about 10,000. The composition has an osmolality in solution that is less than the osmolality of human blood.

### CROSS-REFERENCE TO RELATED APPLICATION

This application is related to and claims priority to U.S. Provisional Application No. 61/021,980 filed Jan. 18, 2008, the entirety of which is incorporated herein by reference.

### STATEMENT REGARDING FEDERALLY SPONSORED RESEARCH OR DEVELOPMENT

n/a

### FIELD OF THE INVENTION

The present invention relates to a composition and method for reducing mental and physical fatigue and increasing energy for athletic performance.

### BACKGROUND OF THE INVENTION

During exercise the body utilizes water, electrolytes, and carbohydrates to operate the musculoskeletal system and maintain homeostatic conditions. An even greater metabolic demand for water, electrolytes, and carbohydrates may be placed on the body when exercise is performed in hot temperatures and the body perspires to maintain homeostasis. In addition to water, perspired sweat contains a variety of electrolytes primarily comprising sodium, chloride, potassium, bicarbonate, and low concentrations of other ions. Electrolyte balance is essential for maintaining precise osmotic gradients, which affect and regulate the hydration of the body, blood pH, and are critical for nerve and muscle tissue. As such, replenishing body fluids during exercise with water alone may result in a rise in blood plasma volume, a fall in sodium level, and a reduction in the osmolality of the blood. This may lead to increased urine production, dehydration, and a reduction in thirst. Dehydration may result in muscle fatigue, lack of sweating, and in severe cases delirium or unconsciousness.

In addition, physical activity places an extra demand on the body's carbohydrate stores, such as muscle glycogen, liver glycogen and plasma glucose. The combination of the loss of water, loss of electrolytes, and the depletion of endogenous carbohydrate stores, are primary causes of fatigue and muscle cramping. This may impair and reduce the body's ability to perform exercise at a high level, both physically and mentally.

However, if carbohydrates, electrolytes, and water are timely replenished during physical exertion, fatigue may be overcome. For example, fluid replacement from sports drinks such as Gatorade® and Powerade® may prevent dehydration and facilitate physical performance. But, these sports drinks utilize low molecular weight simple sugars, such as glucose, sucrose, fructose, or high fructose corn syrup (HFCS), which may be harmful to people with insulin deficiencies, sensitive stomachs, or diabetes. Fructose may cause water retention in the intestine, leading to bloating, excessive flatulence, loose stools, and even diarrhea. HFCS may be further linked to insulin resistance and elevated triglyceride levels. In addition, some sports drinks contain citric acid, which can have a corrosive effect on the teeth of children and adults.

Since athletes often train more than once a day, it is crucial to maximize the rate and efficacy of carbohydrate replenishment. It is just as important to ensure that the delivery method places the least stress on the athlete's digestive system. Taking such considerations allows for the body's resources to be directed toward optimal athletic performance by preventing both muscle and mental fatigue. The body is constantly undergoing protein breakdown and protein building at all times, known as the anabolic and catabolic states. The net protein synthesis is the net summation of the two processes. As an athlete, it is essential to establish an anabolic and anti-catabolic state for prime performance and recovery. It is therefore recommended to intake amino acids or protein to ensure a net positive gain in muscle mass, because exercise quickly generates protein breakdown in the muscles. In addition, recent research has found that certain amino acids can also dramatically increase protein synthesis and delay fatigue. Utilizing these strategies in combination with a surplus of calories may lead to an increase in lean body mass. In some cases, where muscle mass is not desired, such as in marathon runners, protein intake still benefits an athlete due to the accompanying large insulin increase from the combination of carbohydrate and protein intake, as compared to carbohydrates alone. Unfortunately, adding protein to sports drinks tends to make the drink sticky and “thick,” resulting in an uncomfortable feeling of fullness, making it difficult to combine with exercise. Protein hydrolysates may circumvent both of these issues.

Therefore, what is needed is a safe and effective sports drink that maximizes the absorption of carbohydrates, protein, and other nutrients, while providing fluid replenishment, which maximizes muscle recovery and prevents mental fatigue.

### SUMMARY OF THE INVENTION

The present invention relates to a composition and method for reducing fatigue and increasing energy. The composition having a carbohydrate with an average molecular weight greater than 10,000 and a hydrolyzed protein having an average molecular weight less than 10,000.

In another embodiment of the present invention, the composition is an aqueous solution having a carbohydrate with an average molecular weight greater than about 250,000. A hydrolyzed protein is also included, the protein having an average molecular weight less than about 1,000. The osmolality of the composition in solution is less than the osmolality of human blood.

In yet another embodiment of the present invention, the method includes providing an orally administerable aqueous composition having a carbohydrate with an average molecular weight greater than about 10,000. The composition further including a hydrolyzed protein having an average molecular weight less than about 1,000. The osmolality of the composition in solution is less than the osmolality of human blood.

### DETAILED DESCRIPTION OF THE INVENTION

The present invention includes a composition comprising a high molecular weight (HMW) carbohydrate. The HMW carbohydrate may have an average molecular weight greater than about 10,000. For example, as shown in Table 1, the HMW carbohydrate may have an average molecular weight greater than about 10,000, compared to simple sugars and starch syrups, which have a molecular weight below 1,000. The osmolality of the composition in solution having the HMW carbohydrate may be less than the osmolality of human blood. For example, the HMW carbohydrate may have an osmolality less than 200 mOsm/kg in a 15% solution or below 20 mOsm/kg in a 5% solution. The dry weight of the HMW carbohydrate in the composition may be, for example, from 5%-95% of the dry weight of the solids in the composition, or any other percentage. The high molecular weight and low osmolality of the HMW carbohydrate allow for the addition of components into the composition, which may maintain the osmolality of the solution in a hypotonic state. The HMW carbohydrate may be prepared, processed, polymerized, treated, or pre-treated by any methods known to one of ordinary skill in the art.

TABLE 1
Examples of Carbohydrates (MW):

| | |
|---|---|
| Starch | >250,000,000 |
| HMW Carbohydrate | >10,000 |
| Maltodextrin | 1,000-10,000 |
| Starch syrup | 250-1,000 |
| Dextrose | 180 |

The HMW carbohydrate may provide energy, facilitate glycogen storage, or stimulate insulin release. The HMW carbohydrate also may provide for fast glycogen replenishment due to its high molecular weight and low osmolality. Faster intestinal absorption may further be achieved in comparison to low molecular weight carbohydrates, because the HMW carbohydrate may traverse the stomach rapidly due its high molecular weight. The HMW carbohydrate may further promote hydration by reducing water absorption from the body for digestion, and beneficially increase the water supply into the blood stream. The hypotonic nature of the HMW carbohydrate, when compared to isotonic or hypertonic low molecular weight carbohydrates, may cause water to flow from the composition, rather than flow into the composition when being digested. The HMW carbohydrate may further promote an anabolic or anti-catabolic state by increasing insulin production, promoting glycogen replenishment, or minimizing physical fatigue. The high molecular weight and low osmolality of the carbohydrate may further prevent or minimize stomach discomfort by maximizing gastric emptying and intestinal absorption, when compared to low molecular weight sugars.

The composition may further include a hydrolyzed or predigested protein. The hydrolyzed protein may be pretreated with enzymes, or broken down into smaller peptides by the addition of enzymes such as proteases. The term “hydrolyzed” further includes proteins that are, for example, prepared, processed, polymerized, treated, pre-treated, or lysed into smaller peptides or amino acids, by any methods known to one of ordinary skill in the art.

The hydrolyzed protein may comprise, for example, 10-45% of the dry weight of the solids in the composition, or any other percentage. For example, the average molecular weight of 50% of the hydrolyzed protein may be less than about 10,000, or any other percentage or molecular weight. Table 2 shows an example of the molecular weight distribution for a hydrolyzed protein as contemplated by the present invention, based on the percentage of hydrolyzed protein.

TABLE 2
Examples of Molecular Weights of proteins
based on the percentage of hydrolyzed protein (MW):

| Molecular Weight | Percentage (%) |
|---|---|
| >20,000 | 9.5 |
| 5,000-20,000 | 6.6 |
| 1,000-5,000 | 12.9 |
| <1,000 | 70.8 |

The low molecular weight of the protein may facilitate rapid absorption in the digestive system. The hydrolyzed protein may further stimulate greater insulin release in combination with the HMW carbohydrate for faster absorption of nutrients. Because the hydrolyzed protein may be predigested, the body may be able to absorb the protein rapidly and easily during digestion. Moreover, the hydrolyzed protein may further promote an anabolic/anti-catabolic state by increasing both insulin and blood plasma amino acid levels.

A branch chain amino acid (BCAA), for example, Leucine, Isoleucine, or Valine, may be included in the composition. For example, the composition may include all three BCAAs in a ratio of 2:1:1 or 4:1:1 of Leucine, Isoleucine, to Valine. One of ordinary skill in the art will readily appreciate that other ratios may be provided. The concentration of BCAAs in the composition may be from 5-30% of the dry weight of the solids, or any other percentage. The BCAAs may stimulate insulin release in combination with the HMW carbohydrate and the hydrolyzed protein. The BCAAs may further stimulate protein synthesis, muscle recovery, and may prevent fatigue. In particular, Leucine increases protein synthesis by increasing the activity of mTOR & the phosphorylation of eIF4G. Leucine may have a far greater stimulatory effect on protein synthesis than any other amino acid.

Because some neurotransmitters are derived from amino acids, supplementation with BCAAs may increase neurotransmitter production. Exercise reduces plasma BCAA levels, while increasing tryptophan levels. In turn, tryptophan, which is a precursor to serotonin, depresses the Central Nervous System (CNS), leading to fatigue. Unlike other amino acids, which are metabolized in the liver, BCAAs circulate and compete for absorption into the blood-brain barrier. BCAAs compete with the amino acids tryptophan, tyrosine, phenylalanine, and methionine, for access to the neural amino acid transport system, which allows amino acid entry to the brain. Due to the competition between BCAAs and tryptophan, BCAAs may delay CNS fatigue in athletes.

A bicycloheptane (BCH) may also be included in the composition. For example, 2-aminobicyclo-[2,2,1]-heptane-2carboxylic acid, a BCH, may have a concentration from 1-20% of the dry weight of the solids, or any other percentage. BCH is a fatigue recovering agent and fatigue preventative agent for the CNS.

In addition to a hydrolyzed protein, amino acids, such as Glutamine may also be included in the composition. The concentration of the Glutamine in the composition may range from 1-20% of the dry weight of the solids, or any other percentage. Glutamine may cause a significant increase in muscle glycogen storage, which increases the body's capacity to access energy. Glutamine may also be an anti-catabolic agent, which prevents the breakdown of muscle tissue.

Electrolytes, for example, potassium, magnesium, chloride, or sodium may also be included in the composition having a concentration, for example, of 1-5% of the dry weight of the solids, or any other percentage. Electrolytes enable the composition to empty quickly from the stomach and promote absorption from the small intestine. Potassium may be included for optimal muscle contractions and electrolyte replenishment. Magnesium may be included for avoidance of cramping and electrolyte replenishment. Chloride may be included for electrolyte replenishment. Sodium, in combination with natural sweeteners, may be included for electrolyte replenishment or to trigger the body's thirst mechanism, which may prevent dehydration.

In addition to the above components, sweeteners, such as flavoring agents may be included in the composition to increase the palatability of the composition. Other components, such as minerals or vitamins such as Vitamin A, Vitamin B, Vitamin C, Vitamin E, or Vitamin D may also be included to provide beneficial nutrients in addition to the components that prevent physical and mental fatigue. Creatine may also be included in the composition, and may have an increased absorption in combination with the HMW carbohydrate and hydrolyzed protein. Beta-alanine may also be included in the composition and may increase carnosine levels in the muscle, which may allow the muscles to work longer without fatigue. Antioxidants may also be included in the composition in varying concentrations to improve the overall efficacy of the composition. The HMW and low osmolality of the carbohydrate combined with other nutrients may result in nutrient absorption that coincides with a corresponding insulin spike, resulting in optimal nutrient delivery. In contrast, the combination of nutrients with conventional carbohydrates results in an insulin response, but with a delay in nutrient absorption, resulting in suboptimal delivery.

Any of the above components may be administered orally in a liquid mixture form, for example, as a sports drink or aqueous solution. Alternatively, the above components may be administered in pill, capsule, tablet, powder, gel tabs, or other solid forms. One of ordinary skill in the art will readily appreciate that other forms of administration that may be provided. It is further contemplated that the composition may include the isomers, salts, or derivatives of any of the components described herein.

The examples below are formulations of the composition of the present invention. The examples are exemplary, and do not limit the invention to the embodiments illustrated in the examples below.

| | Percentage |
|---|---|
| Component | (weight/volume) |
| HMW Carbohydrate (>10,000, | 50-90% |
| <200 mOsm/Kg in 15% solution) | |
| Hydrolyzed Protein (over 50% <1,000) | 10-45% |
| Leucine | 5-30% |
| BCH | 1-20% |
| Glutamine | 1-25% |
| Electrolytes | 1-5% |
| Sweeteners | 1-5% |

### EXAMPLE 1

| | Percentage |
|---|---|
| Component | (weight/volume) |
| HMW Carbohydrate (>500,000, | 50-90% |
| <100 mOsm/Kg in 15% solution) | |
| Hydrolyzed Protein (over 75% <10,000) | 10-45% |
| Leucine | 5-30% |
| BCH | 1-20% |
| Glutamine | 1-25% |
| Electrolytes | 1-5% |
| Sweeteners | 1-5% |
| Creatine | 1-5% |
| Antioxidants | 1-5% |
| Beta-alanine | 1-5% |

### EXAMPLE 2

| | Percentage |
|---|---|
| Component | (weight/volume) |
| HMW Carbohydrate (>500,000, | 50-90% |
| <50 mOsm/Kg in 15% solution) | |
| Hydrolyzed Protein (over 25% <1,000) | 10-45% |
| Leucine | 5-30% |
| BCH | 1-20% |

### EXAMPLE 3

It will be appreciated by persons skilled in the art that the present invention is not limited to what has been particularly shown and described herein above. A variety of modifications and variations are possible in light of the above teachings without departing from the scope and spirit of the invention, which is limited only by the following claims.

What is claimed is:

1. A composition for reducing fatigue and increasing energy, comprising:
a carbohydrate with an average molecular weight greater than about 10,000; and
a hydrolyzed protein having an average molecular weight less than about 10,000.

2. The composition of claim 1, wherein the osmolality of the composition in solution is less than the osmolality of human blood.

3. The composition of claim 2, wherein the average molecular weight of the carbohydrate is greater than about 100,000.

4. The composition of claim 2, further comprising a branch chain amino acid.

5. The composition of claim 4, wherein the branch chain amino acid is at least one of:
(a) Leucine,
(b) Isoleucine,
(c) Valine, or
(d) salts, isomers, or derivatives of at least one of (a), (b), and (c).

6. The composition of claim 2, further comprising a bicycloheptane (BCH).

7. The composition of claim 2, further comprising Glutamine, or salts, isomers, or derivatives of Glutamine.

8. The composition of claim 2, further comprising at least one vitamin.

9. The composition of claim 2, further comprising at least one sodium, potassium, or chlorine electrolyte.

10. The composition of claim 2, further comprising beta-alanine, or salts, isomers, or derivatives of beta-alanine.

11. The composition of claim 2, further comprising at least one antioxidant.

12. The composition of claim 2, further comprising creatine, or salts, isomers, or derivatives of creatine.

13. The composition of claim 1, wherein the carbohydrate comprises approximately 25%-95% of the dry weight of the solids.

14. A composition for reducing fatigue and increasing energy, comprising:
an aqueous solution having a carbohydrate with an average molecular weight greater than about 250,000;
a hydrolyzed protein within the aqueous solution having an average molecular weight less than about 1,000; and wherein the composition has an osmolality in solution less than the osmolality of human blood.

15. The composition of claim 14, further comprising Glutamine, or salts, isomers, or derivatives of the same.

16. The composition of claim 14, further comprising:
(a) Leucine,
(b) Isoleucine,
(c) Valine, or
(d) salts, isomers, or derivatives of at least one of (a), (b), and (c).

17. The composition of claim 14, further comprising a bicycloheptane, or salts, isomers, or derivatives of bicycloheptane.

18. The composition of claim 14, wherein the carbohydrate comprises approximately 25%-95% of the dry weight of the solids.

19. A method for preventing fatigue and increasing energy, comprising:
providing an orally administerable aqueous composition comprising a carbohydrate having an average molecular weight greater than about 10,000, the composition further comprising a hydrolyzed protein having an average molecular weight less than about 10,000, and wherein the composition has an osmolality in solution less than the osmolality of human blood.

20. The method of claim 19, wherein the average molecular weight of the carbohydrate is greater than about 250,000.
